(19) United States
(12) Patent Application Publication
(10) Pub. No.: US 2008/0112995 A1
(43) Pub. Date: May 15, 2008

(54) Implantable Bioreactors and Uses Thereof

(75) Inventor: Alon Shalev, RaAnana (IL)

Correspondence Address: Martin D. Moynihan;PRTSI, Inc., P.O. Box 16446, Arlington, VA, 22215, US

(73) Assignee: Nicast Ltd., Lod (IL)

(21) Appl. No.: 11/795,914

(22) PCT Filed: Jan. 25, 2006
(86) PCT No.: PCT/IL06/00103

§ 371 Date: Jul. 24, 2007

Related U.S. Application Data
(60) Provisional application No. 60/646,543, filed on Jan. 25, 2005.

(51) Int. Cl. A61F 2/00; A61K 35/12; A61K 31/727; A61K 31/44; A61K 31/425; A61P 43/00

(52) U.S. Cl. 424/423; 424/93.7; 514/56; 514/301; 514/365

(57) ABSTRACT

An implantable bioreactor device and methods of use are provided. The device comprises a first compartment being configured capable of fluidic communication with a vasculature of a subject; and a second compartment configured for containing cells, said second compartment being separated from said first compartment by a membrane.

### FIELD AND BACKGROUND OF THE INVENTION

The present invention relates to a device capable of maintaining cells under an immunoprivileged, vascularized environment. More specifically, the present invention relates to a bioartificial organ, such as a pancreas and to methods of implanting and utilizing same for treating disorders associated with organ deficiencies or failure.

Treating pathologies requiring a continuous supply of biologically active substance has made necessary the production of implantable bioreactor devices able to efficiently release such biologically active substances over extensive periods of time. Such bioreactors are, for example, bioartificial organs containing cells producing one or more biologically active substance of interest. The cells contained in a bioartificial organ are enclosed in internal spaces or encapsulation chambers bound by at least one semi-permeable membrane. Such a semi-permeable membrane should allow the biologically active substances of interest to pass, which should be available to the target cells aimed at in the patient's body, while being impermeable to the patient's cells, more particularly to the immune system cells, as well as to antibodies and other toxic substances.

A bioartificial pancreas refers to a system incorporating beta cells (usually in the form of Islets of Langerhans) of either human or animal origin, typically implanted within the interstitial space and protected by a semi-porous membrane. The abovementioned membrane enables the beta cells to maintain normal metabolism, sense interstitial glucose levels, secrete insulin in correlation with sensed glucose levels, yet sustain the cells within an immunoprivileged environment.

However, encapsulated cell implants often suffer from a poor supply of nutrients and/or removal of metabolites from the implants themselves. This commonly leads to encapsulated cell necrosis and reduced production of cellular products.

The impact of hypoxia is also influenced by the type of cells/tissues being implanted. Thus, for example, pancreatic islet cells are especially prone to oxygen supply limitations because they have a relatively high oxygen consumption rate. They are normally highly vascularized and are supplied blood at arterial pO₂. When cultured in vitro under normoxic conditions, islets develop a necrotic core, the size of which increases with increasing islet size, as is to be expected as a result of oxygen diffusion and consumption within the islet. However, the death of implanted cells due to hypoxia is not the only concern. Oxygen levels high enough to keep cells alive can nonetheless have deleterious effects on cell functions that require higher cellular ATP concentrations, for example, ATP-dependent insulin secretion.

With few exceptions, only by suspending islets in an extracellular gel matrix at very low islet volume fractions (e.g., 1 to 5%), which greatly increases the size of the implanted device, have investigators been able to maintain the viability of the initially loaded islets. However, use of such low tissue density puts undesirable constraints on the maximum number of islets that can be supported in a device of a size suitable for surgical implantation.

Attempts to modify the design of bioreactor devices have been made to try to overcome these limitations. A biohybrid artificial pancreas for insulin secretion known in the art consists of a semipermeable membrane tube through which arterial blood flows. The membrane tube is surrounded by the implanted tissue which is, in turn, contained in a housing. This approach provides the highest available pO₂ (100 mm Hg) but suffers from the need to open the cardiovascular system; thus, it may be limited to only a small fraction of patients.

One alternative is an extravascular device in the form of a planar or cylindrical diffusion chamber implanted, for example, in subcutaneous tissue or intraperitoneally. Such devices are exposed to the mean pO₂ of the microvasculature (about 40 mm Hg) limiting the steady state thickness of viable tissue that can be supported. Further limits are imposed when such devices are implanted into soft tissue. If a foreign body response occurs, an avascular fibrotic tissue layer adjacent to the chamber can be produced, typically on the order of 100 μm thick. This fibrotic tissue increases the distance between blood vessels and implant, and the fibroblasts in fibrotic tissue layer also consume oxygen. Oxygen deficits are especially likely during the first few days following implantation before neovascularization has a chance to occur. Anoxia may exist within regions of the device, leading to death of a substantial fraction of the initially implanted tissue.

Microporous membranes that induce neovascularization at the device-host tissue interface have also been used. This angiogenic process takes 2-3 weeks for completion, and the vascular structures induced remain indefinitely. By bringing some blood vessels close to the implant, oxygen delivery is improved. Oxygen delivery also may be improved by prevascularizing the device, e.g. by infusion of an angiogenic factor(s) through the membranes into the surrounding tissue.

Another means of implanting cells in an extravascular environment involves the use of spherical microcapsules. The microcapsules comprise small quantities of cells enclosed in a semipermeable membrane and can be implanted in an extravascular space, for example, in the peritoneal space. However, the large volume of microcapsules employed, and the tendency for most to permanently attach to peritoneal surfaces, may lead to clinical problems. Thus, despite encouraging results with various tissues and applications, the problems of oxygen transport limitations remain.

There is thus a widely recognized need for, and it would be highly advantageous to have a bioreactor device which allows cells housed within to be exposed to high concentrations of oxygen, whilst maintaining an immunoprivileged environment.

### SUMMARY OF THE INVENTION

According to one aspect of the present invention there is provided a bioreactor device comprising a first compartment being configured capable of fluidic communication with a vasculature of a subject and a second compartment configured for containing cells, the second compartment being separated from the first compartment by a membrane.

According to another aspect of the present invention there is provided a method of delivering a cell population into a subject in need thereof, the method comprising providing a device comprising a first compartment being configured capable of fluidic communication with a vasculature of a subject and a second compartment configured for containing cells, said second compartment being separated from said first compartment by a membrane; connecting the device to the vasculature of the subject and introducing the cell population into the second compartment, thereby delivering a cell population into a subject in need thereof.

According to further features in preferred embodiments of the invention described below, the membrane blocks passage of said cells from said second compartment to said first compartment.

According to still further features in the described preferred embodiments the membrane enables passage of fluids and molecules to and from the second compartment.

According to still further features in the described preferred embodiments the first and second compartments are housed within a device body configured suitable for implantation into the subject.

According to still further features in the described preferred embodiments the first compartment comprises a blood inport and a blood outport.

According to still further features in the described preferred embodiments the first compartment further comprises a vascular prosthesis connected to each of the blood inport and the blood outport.

According to still further features in the described preferred embodiments the first compartment includes a cell injection port.

According to still further features in the described preferred embodiments the first compartment or each of the blood inport and blood outports or the vascular prosthesis is configured such that when the bioreactor device is connected to a vasculature of the subject, a blood pressure at the blood inport is higher than a blood pressure at the blood outport.

According to still further features in the described preferred embodiments the blood pressure is reduced with minimum blood turbulence.

According to still further features in the described preferred embodiments at least one of the first compartment, the second compartment and the membrane is made of non-woven polymer fibers.

According to still further features in the described preferred embodiments the non-woven polymer fibers are electrospun polymer fibers.

According to still further features in the described preferred embodiments the first compartment and/or the membrane comprise at least one pharmaceutical agent.

According to still further features in the described preferred embodiments the at least one pharmaceutical agent is impregnated in the vascular compartment and/or the membrane.

According to still further features in the described preferred embodiments the pharmaceutical agent is a therapeutic agent or a diagnostic agent.

According to still further features in the described preferred embodiments the therapeutic agent is selected from the group consisting of heparin, tridodecylmethylammonium-heparin, epothilone A, epothilone B, rotomycine, ticlopidine, dexamethasone and caumadin.

According to still further features in the described preferred embodiments the polymer fibers have a permeability cutoff at a molecular weight of about between 40 and 250 kilo Daltons.

According to still further features in the described preferred embodiments the step of introducing the cell population into the second compartment is effected prior to the step of connecting the device to the vasculature of the subject.

According to still further features in the described preferred embodiments the step of introducing the cell population into the second compartment is effected following the step of connecting the device to the vasculature of the subject.

According to still further features in the described preferred embodiments the at least one of the first compartment and the second compartment is made of non-woven polymer fibers.

According to still further features in the described preferred embodiments the non-woven polymer fibers are electrospun polymer fibers.

According to still further features in the described preferred embodiments the step of connecting the device to the vasculature of the subject is effected by an end to end vascular connection.

According to still further features in the described preferred embodiments the step of connecting the device to the vasculature of the subject is effected by an end to side vasculature connection.

According to still further features in the described preferred embodiments the step of connecting the device to the vasculature of the subject is effected by a combination of an end to side vasculature connection and an end to end vasculature connection.

According to still further features in the described preferred embodiments the cell population comprises an insulin secreting cell population.

According to still further features in the described preferred embodiments the cell population comprises Islets of Langerhans.

The present invention successfully addresses the shortcomings of the presently known configurations by providing a bioreactor device and a method of delivering a cell population.

### BRIEF DESCRIPTION OF THE DRAWINGS

The invention is herein described, by way of example only, with reference to the accompanying drawing. With specific reference now to the drawing in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fumdamental understanding of the invention, the description taken with the drawing making apparent to those skilled in the art how the several forms of the invention may be embodied in practice.

In the drawings:

FIG. 1 illustrates a bioreactor device according to a preferred embodiment of the present invention.

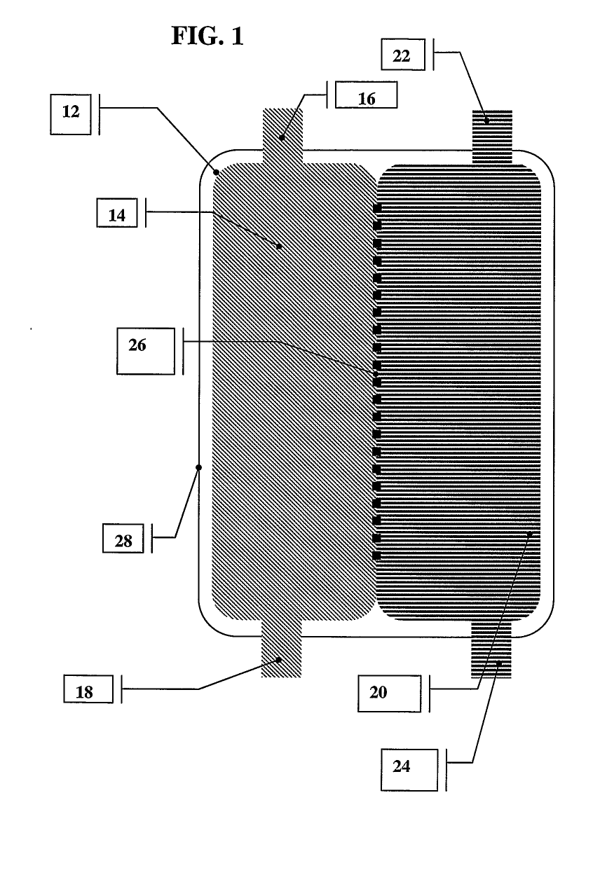

### DESCRIPTION OF THE PREFERRED EMBODIMENTS

The present invention relates to a bioreactor device and methods of implantation such that cells encapsulated within remain in an immunoprivileged, vascularized environment. More specifically, this invention relates to a bioartificial pancreas and methods of implanting and using same.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Numerous types of implantable bioreactors are known in the art. Although the problem of sufficient oxygen exposure to cells housed within such devices has been addressed by some prior art designs, there remains a need for bioreactors which can house cells particularly susceptible to oxygen levels, such as pancreatic islet cells and yet keep such cells in an immunoprivileged environment.

While reducing the present invention to practice, the present inventors have devised a bioreactor configuration which overcomes the limitations of prior art by providing a dual compartment device in which the cell containing compartment is separated from a compartment connectable to a vascular network via a membrane. The compartment connected to the vasculature aids in the provision of nutrients and oxygen to cells housed in the second compartment. The device may be used to grow or maintain cells ex vivo, it may be provided extracorporeally and yet be connected to a subject's vasculature, or it may be implanted and used as an in-body bioreactor for producing molecules (e.g. insulin) and/or cells beneficial to the body.

Thus, according to one aspect of the present invention, there is provided a bioreactor device.

The device of the present invention a first compartment which is configured capable of fluidic communication with a vasculature of a subject and a second compartment which is configured for containing cells. The second compartment is separated from the first compartment by a membrane.

As used herein, the term “bioreactor” refers to an enclosed or partially enclosed device for maintaining cells viable under proliferative or non-proliferative conditions.

The term “vasculature” as used herein refers to the vascular system (or any part thereof) of a body, human or non-human, and includes blood vessels, e.g., arteries, arterioles, veins, venules, capillaries and lymphatics.

As used herein, the term “cells” refers to any cellular matter that may be maintained in a bioreactor. The cells may be individual or isolated cells, cell lines, tissue fragments and/or cell aggregates. Preferably the tissue fragment or cell aggregates do not comprise deep buried cells (i.e. cells that are no more than about a few tens of microns from a surface.) since diffusion of oxygen and nutrients will not be sufficient to maintain them. Preferably, the smallest size of a tissue fragment will not exceed 20 microns. Preferably the cells are of mammalian origin e.g. human or porcine. The cells may come from a variety of organ sources, including, but not limited to, pancreas cells, hepatocytes, kidney cells, lung cells, neural cells, pituitary cells, parathyroid cells, thyroid cells, and adrenal cells. Multiple types of cells may be mixed in the cell containing compartment (e.g., hepatocytes and pancreas cells).

Thus, in the case of insulin secreting bioreactor, the cellular matter that can be included in the second compartment can be an isolated beta cell, an Islet of Langerhan, or a pancreas fragment.

The cell may also be genetically modified so that it is capable of providing a function lacking in a body of a patient. Suitably the cell is modified to have altered gene expression through the introduction of expression vectors comprising a nucleic acid encoding the gene of interest. For example, a hepatic cell may be genetically modified to induce expression of endogenous insulin either directly or indirectly through a cascade of regulatory gene expression events e.g. by the expression of pd-x.

Alternatively a cell may be genetically modified so as to enhance in the grafting process of the implanted bioreactor. For example, a cell may be genetically modified to secrete an angiogenic factor such as VEGF (e.g. NM_{—}003377, NM_{—}00469.2, NM_{—}005429.2 and NM_{—}001025366.1) that may enhance in the vascularization of the bioreactor.

This can proceed, for example, by transfecting cells to secrete angiogenic factor(s). Various methods can be used to introduce the expression vector of the present invention into the host cell system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. Examples of viral vectors include, but are not limited to adenoviral, adeno-associated, retroviral, and lentiviral vectors.

Other angiogenic factors for use with the current invention include but are not limited to Platelet-derived Endothelial Cell Growth Factor, Angiogenin, basic and acidic Fibroblast Growth Factor (also known as Heparin Binding Growth Factor I and It, respectively), Transforming Growth Factor-Beta, Platelet-derived Growth Factor, Hepatocyte Growth Factor, Fibroblast Growth Factor-18, Butyryl Glycerol, prostaglandins PGE1 and PGE2, nicotinamide, Adenosine, (12R)-hydroxyeicosatrienoic acid, and okadaic acid. In a preferred embodiment, the angiogenic factor produced by the transduced cells is VEGF. The human VEGF gene has been used in vivo in several mammalian models for angiogenesis with no immunogeneic response reported. Furthermore, VEGF has been shown to be highly specific, since its receptors are localized almost exclusively in vascular endothelial cells. Naturally, other angiogenic growth factors can be used with the present invention and include other isoforms of vascular endothelial growth factor (VEGF), angiopoietins, fibroblast growth factors (FGF).

The cells may be differentiated or non-differentiated (i.e. stem cells). As used herein, the phrase “stem cells” refers to cells which are capable of remaining in an undifferentiated state (i.e. “pluripotent stem cells”) for extended periods of time in culture until induced to differentiating into other cell types having a particular, specialized function (i.e., “differentiated” cells).

Non-limiting examples of stem cells which can be used in the present device are hematopoietic stem cells obtained from bone marrow tissue of an individual at any age or from cord blood of a newborn individual, embryonic stem (ES) cells obtained from the embryonic tissue formed after gestation (e.g., blastocyst), or embryonic germ (EG) cells obtained from the genital tissue of a fetus any time during gestation, preferably before 10 weeks of gestation. The stem cells of the present invention can also be adult tissue stem cells. As used herein, “adult tissue stem cells” refers to any stem cell derived from the postnatal animal (especially the human). The adult stem cell is generally thought to be a multipotent stem cell, capable of differentiation into multiple cell types. Adult stem cells can be derived from an adult tissue such as adipose tissue, skin, kidney, liver, prostate, pancreas, intestine, and bone marrow.

The stem cells may be differentiated prior to introduction into the second compartment or may be differentiated in the cell compartment. Exemplary conditions for differentiating cells include maintaining under conditions that promote differentiation in a particular manner. Such conditions may include withdrawing or adding nutrients, growth factors or cytokines to the medium, changing the oxygen pressure, or altering the substrate on the culture surface. For example, embryonic stem cells can be induced to differentiate in vitro into cardiomyocytes [Paquin et al., Proc. Nat. Acad. Sci. (2002) 99:9550-9555]. Several factors alone or in combination have been shown to enrich cardiac differentiation such as hepatocyte growth factor (HGF), epidermal growth factor (EGF), basic fibroblast growth factor (bFGF), transforming growth factor β1(TGF β1), platelet derived growth factor (PDGF), sphingosine-1-phosphate, retinoic acid, 5-azacytidine and vitamin C. Embryonic stem cells have also been induced to differentiate into neural or glial lineages [Reubinoff et al., Nature Biotechnology (2001) 19:1134-1140; U.S. Pat. No. 5,851,832]. For their generation, the medium typically includes any of the following factors or medium constituents in an effective combination: Brain derived neurotrophic factor (BDNF), neutrotrophin-3 (NT-3), NT-4, epidermal growth factor (EGF), ciliary neurotrophic factor (CNTF), nerve growth factor (NGF), retinoic acid (RA), sonic hedgehog, FGF-8, ascorbic acid, forskolin, fetal bovine serum (FBS), and bone morphogenic proteins (BMPs). Embryonic stem cells have also been induced to differentiate into hematopoietic cells [Weiss et al., Hematol. Oncol. Clin. N. Amer. (1997) 11(6): 1185-98; U.S. Pat. No. 6,280,718] and insulin-secreting beta cells [Assady et al., Diabetes (2001) 50(8):1691-1697].

Differentiation of stem cells can also be directed by genetic modification. Several transcription factors have been demonstrated to regulate differentiation of ES cells to specific cell types [Levinson-Duslinik M., Benvenisty N., Cell Biol. 17: 3817-3822, 1997]. Ectopic over-expression of such factors stimulates ES cells to differentiate selectively into certain cell types. For example over-expression of the transcription factor GATA-4 was shown to induce cardiomyocyte differentiation [Grepin C., et al., Development 124: 2387-2395, 1997; Fujikura J., et al., Genes Dev. 16: 784-789, 2002; Kanda S., et al., Hepatol. Res. 26:225-231, 2003].

The cells or tissue used by the present device may be suspended in a liquid trapped within the second compartment, adhered to the inner walls of the compartment or immobilized on an appropriate support structure provided within the compartment. For example, the cells may be embedded in a gel matrix (e.g., agar, alginate, chitosan, polyglycolic acid, polylactic acid, and the like). Alternatively, cells may be seeded over a porous scaffold (e.g. an alignate scaffold). In another embodiment, cells in the cellular compartment may themselves be encapsulated within microcapsules or attached to beads.

The number of cells required for transplantation may be determined by the secretion rate of the desired agent by the cell and the amount of active agent required by the body. For example, the average patient with IDDM requires approximately 30 Units of insulin per day to control blood glucose levels. The amount of insulin (Units/cells/day) produced by a population of islet cells is then determined in culture and the number of transplanted cells needed to provide the patient's required insulin dose is loaded within the second compartment. Typically, this is on the order of about 10⁹ cells. In the case of cells that proliferate, a determination of the insulin production rate in culture permits an estimation of the number of cells required in a given volume within the second compartment. A small number of cells can be used which will then proliferate to fill the volume of the compartment and provide the necessary amount of insulin. It is readily apparent, that similar calculations may be performed for any application for which a required dosage of active agent is known, or is determinable, and for which the amount of bioactive agent produced by cells may be measured.

As is mentioned hereinabove, the first and second compartments of the present device are separated by a membrane. The membrane utilized by the present invention is typically semi-permeable. Minimally the membrane must be have a pore size, pore density, percent porosity, molecular weight cut off to keep cells within the cellular compartment. Particularly in embodiments where allergenic or autologous cells are transplanted, the pore size may be quite large as about 0.1 μm but must completely prevent passage of cells. In the embodiment of the invention where the cells within the cellular compartment are xenogeneic, the membrane should have a pore size, pore density, percent porosity, molecular weight cutoff, sufficient to keep selected components of the immune system out, yet allowing for the transport of nutrients, oxygen, secreted cellular products (e.g., secreted insulin), metabolic wastes, ions, and other bioactive molecules. When xenogeneic cells are used, the molecular weight cutoff should be about 100,000 daltons or less, so as to prevent components of the humoral immune system from entering into the cellular compartment and to prevent endogenous retroviruses transmittable by the cells, or other infectious macromolecules which might be secreted by the cells, from exiting the cellular compartment.

Alternatively, the membrane may allow a selective cell passage from the cellular compartment to the vascular compartment, such that smaller cells e.g. stem cells pass through the membrane, and larger differentiated cells are retained within the cellular compartment.

Semi-permeable materials that may be used to fabricate the membrane are described hereinbelow.

The first compartment (i.e. vascular compartment) preferably comprises a blood inport and a blood outport such that it is capable of fluid communication with the vasculature of a subject. The first compartment may also comprise a vascular prosthesis connected to the blood inport and the blood outport.

As used herein, the term vascular prosthesis refers to any tubular structure which is suitable for use, for example, as a vascular graft.

According to one embodiment the blood import, blood outport and vascular prosthesis are configured in such a way that when the bioreactor device is connected to the vasculature of a subject, the blood pressure at the inport is higher than the blood pressure at the outport. This ensures a flow of blood through the blood compartment allowing maximal diffusion of oxygen and nutrients from the vascular compartment to the cell containing compartment and maximal diffusion of waste products from the cell containing compartment to the blood compartment. Preferably, the blood inport and blood outport have an inner diameter of at least 2 mm, more preferably 4-8 mm and even more preferably about 6 mm. Preferably, the tensile strength of the blood inport and export is at least 10 N so as to discourage rupturing of the connections. The lengths of the cell injection inport and outport depend on the loci of implantation relative to the loci of positioning the cell injection ports on the body surface. Typically, the length is from 2 cm to 20 cm.

The vascular prosthesis aids in reducing blood pressure at the blood outport by providing an extensive vascular meshwork through which the blood must travel in order to reach the outport. Preferably the vascular prosthesis aids in pressure reduction with minimal blood turbulence. The dimensions of the prosthesis are: length: 5-50 nm (preferably—30 mm); internal diameter: 2-8 mm diameter (preferably—5-6 mm); external diameter: 1-4 mm greater than the internal diameter.

As mentioned herein above, the blood inport and blood outport of the device of the present invention may be connected to the vasculature of a subject. Any combination of vasculature to blood inport or blood outport is envisaged. For example, the blood inport may be connected to an artery, and the blood outport may be connected to a vein. Alternatively, the blood inport may be connected to a vein and the blood outport to either an artery or a vein. In a preferred embodiment, the blood inport of the device of the present invention is connected to an artery and the blood outport of the device is connected to a vein. Using this configuration, it is expected that dissociation between oxygen and erythrocytes within the vascular compartment is enhanced, thus providing more oxygen available for nourishing the grafted cells.

Connection of the blood inport and blood outport to the vasculature may be effected using an end-to-end connection, an end-to-side (T-junction) connection or a combination of both. The bioreactor of the present invention may be connected to the vasculature using sutures, staples or clamps. Such clamps are disclosed in U.S. Pat. Nos. 3,357,432; 3,435,823 and 6,402,767. Another vascular prosthesis connector is disclosed in FR2683141 by Thierry Richard and Eric Perouse entitled “Connection device for organ vessel prostheses.” Other agents that aid in sealing the blood inport to the vasculature that may be used in the context of the present invention include various biological glues.

The bioreactor device of the present invention including the cellular compartment, vascular compartment, membrane and housing, can be fabricated from a biodegradable, a biostable polymer or a combination of a biodegradable and a biostable polymer.

Suitable biostable polymers which can be used in the present embodiments include, without limitation, polycarbonate based aliphatic polyurethanes, silicon modificated polyurethanes, polydimethylsiloxane and other silicone rubbers, polyester, polyolefins, polymethyl- methacrylate, vinyl halide polymer and copolymers, polyvinyl aromatics, polyvinyl esters, polyamides, polyimides and polyethers.

Suitable biodegradable polymers which can be used in the present embodiments include, without limitation, poly (L-lactic acid), poly (lactide-co-glycolide), polycaprolactone, polyphosphate ester, poly (hydroxy-butyrate), poly (glycolic acid), poly (DL-lactic acid), poly (amino acid), cyanocrylate, some copolymers and biomolecules such as collagen, DNA, silk, chitozan and cellulose.

It is expected that during the life of this patent many relevant polymeric material will be developed and the scope of the term polymer is intended to include all such new technologies a priori.

The bioreactor device of the present invention can be a variety of shapes including tubes or cylinders, cubes, spheres, discs, or sheets, so long as it is able to provide a sufficient containment surface for the number of cells suitable for a given application and a sufficient containment surface for the amount of blood needed to oxygenate the cells. Examples of typical shapes and volumes of bioreactor compartments are provided herein below:

Blood Inport, Outport

### Surface Area: 15-40 mm²; preferably—25-35 mm²

### Shape: preferred circular

Cell injection port, Cell flushing port

### Surface Area: 10-30 mm²; preferably—15-25 mm²

### Shape: preferred circular

Vascular compartment

### Volume: 20-4000 mm³; preferably—100-1000 mm³

### Shape: preferably cylindrical

### Length: 5-50 mm

### Diameter: 3-5 mm

Cellular compartment

### Volume: 10-3000 mm³; preferably—10-100 mm³

### Shape: preferably coaxially cylindrical around vascular compartment

Membrane

Membrane surface area: 50-1500 mm²

The cellular compartment may be coated by biocompatible molecules on the interior (i.e., the side proximal to the encapsulated cells), such as polymeric scaffolds or gel matrices (as described hereinabove) which may also be coated by bioactive molecules such as ECM proteins, morphogenic proteins, growth factors, cytokines, and/or polysaccharides.

The components of the bioreactor may also be coated with an anti-microbial and/or an anti-thrombotic agent on its outside surface. In addition, the bioreactor may be coated with agents that prevent biofilm formation or agents that aid in the reduction of an immunogenic response.

The present inventor has postulated that a porous, open cell matrix and biostable matrix will be able to provide free transport of nutrients (e.g. oxygen, glucose) and metabolites (e.g. CO₂) into and out from the cellular compartment and thus support its sustained vitality, while at the same time, will provide an immunoprivileged environment by preventing cellular components from infiltrating the cellular compartment (i.e. cells of immune system) and invoking a host response againt the non-autologous cellular components. Thus, polymer fibers, specifically non-woven polymer fibers (preferably electrospun) may provide fabrication for the bioreactor device of the present invention.

Thus, the membrane, vascular compartment and/or cell compartment may all be fabricated from electrospun fibers. By selecting a polymer fiber of a particular permeability cut-off, a membrane fabricated therefrom can be selective for particular nutrients and molecules. Typically, the polymer fibers have a permeability cutoff at a molecular weight of about between 40 and 250 kilo Daltons. Thus the permeability cutoff may be for example about 40 kilo Daltons, 60 kilo Daltons, 80 kilo Daltons, between about 100-150 kilo Daltons or between about 150-250 kilo Daltons.

The present inventor has shown that vascular prosthesis may be fabricated with electrospun fibers since these provide an exceptionally good interface for physiological integration between an artificial vascular prosthesis and biologic vasculatures. Moreover, the present inventor has found that electrospun vascular grafts have inherent self-sealing properties, due to the elasticity of the micrometric or sub-micrometric arrangement of the elastomeric matrix. Thus, electrospun fibers may be particularly useful for fabricating the blood inport, blood outport and vascular prosthesis of the vascular compartment of the bioreactor device of the present invention.

Thus, the bioreactor device of the present embodiments is preferably wholly or partially fabricated using an Electrospinning approach. The Electrospinning steps may be performed using any Electrospinning apparatus known in the art. Suitable Electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference. Other spinning techniques are disclosed, e.g., U.S. Pat. Nos., 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

Reinforcing fibers can be made from monofilaments of polymers such as PTFE, PET, PEN and customarily created in the form of a wound coil. Such monofilaments may have a typical diameter between 0.1 mm and 1 mm, preferably around 0.5 mm.

The bioreactor device of the present embodiments can also include pharmaceutical agents selected in accordance with the application and expected pathology. For example, the implantation of the bioreactor may result in disorders such as immune rejection and hyper cell proliferation. The incorporated pharmaceutical agent can therefore be a medicament for treating such and other disorders. In addition, thrombogenic agents may be particularly useful for including in the blood inport, blood outport and vascular prosthesis of the vascular compartment of the bioreactor device. For example, thrombogenic agents could minimize leakage from needle puncture holes following connection of the bioreactor to a patient's vasculature. Upon needle extraction, thrombogenic agents could trigger a localized coagulation process at the periphery of the needle hole, thereby enhancing the self sealing properties of an electrospun-polymer fabricated bioreactor.

It is recognized that factors which facilitate generation of haemostatic plug include adhesion and aggregation of platelets as well as formation of polymerized fibrin matrix at the site of vascular injury. The endothelial surface on the vessel wall is not thrombogenic. Vascular wall injury results in exposure of collagen and subendothelial proteins. The adherence of platelets to collagen is recognized as a critical initial event for generation of a haemostatic plug. The reason being the capturing of platelets from the flowing blood via rapid bond formation between their glycoprotein 1b receptor and von Willibrand factor immobilized on collagen.

In parallel with the platelets adhesion process, coagulation is initiated through release of tissue factor from the damaged vessel wall. Propagation of blood coagulation occurs by localized enzymatic complexes assembled on the plasma membrane of adherent platelets that expose negatively charged phospholipids. The thrombin thus formed further activates platelets and stabilizes the growing thrombus by the formation of fibrin.

Both platelets that are in direct contact with subendothelial collagen and platelets that form the main body of an adherent platelets thrombus can participate in the clot formation. The direct contact platelets are activated by collagen as well as by soluble agonists (such as thrombin). On the other hand, the platelets of the main body of the thrombus are activated by soluble agonists, with minimal or no collagen impact. According to a preferred embodiment of the present invention the thrombogenic agent is selected to affect the first phase of the thrombus formation so as to create weak clot formation and to occlude the holes in the artificial vessel.

Representative examples for suitable thrombogenic agents include, thrombin, a platelet activating factor or an analogue thereof, fibrin, factor V, factor IX, an antiphospholipid antibody or a portion thereof, copper or an alloy thereof, platinum or an alloy thereof a positively charged polymer (voltage being in the range between 0.2 and 0.8 volts), polyvinyl acrylate and cyanoacrylate.

Such agents are readily available in active or precursor form from a variety of suppliers. For example, thrombin or prothrombin can be obtained from Sigma-Aldrich.

According to presently preferred embodiments of the present invention, the thrombogenic agent is collagen (available from Sigma and BD Biosciences), von Willebrand Factor (preferably from a human source from HTI and American Diagnostica), thrombospondin (available from ProSpecTany TechnoGene and Sigma), tissue factor (available from Dade Behring and American Diagnostica), or various phospholipids (e.g. L-alpha Phosphatidylcholine, L-alpha-Phosphatidylserine, L-alpha-Phosphatidylethanolamine available from Avanti polar lipids)

Additionally or alternatively, the incorporated pharmaceutical agent can be an imaging contrast agent to enable post implantation imaging. The additional pharmaceutical agent can be coated upon, attached to or impregnated within any layer of the tubular structure. Further details of various approaches suitable for coating, impregnating or modifying polymers with various agents can be found, for example, in WO 02/049536 and WO 02/49535, supra.

Examples of therapeutic agents which may be used according to this aspect of the present invention, include, but are not limited to an antithrombotic agent, an estrogen, a corticosteroid, a cytostatic agent, an anticoagulant, a vasodilator, an antiplatelet agent, a thrombolytic agent, an antimicrobial agent, an antibiotic, an antimitotic, an antiproliferative agent, an antisecretory agent, a non-sterodial anti-inflammatory agent, a growth factor antagonist, a free radical scavenger, an antioxidant, and an immunosuppressive agent.

Particularly preferred therapeutic agents which may be used according to this aspect of the present invention include, but are not limited to heparin, tridodecylmethylammonium-heparin, epothilone A, epothilone B, rotomycine, ticlopidine, dexamethasone and caumadin.

Examples of contrast imaging agents which may be used in accordance with this aspect of the present invention include, but are not limited to an X-ray imaging contrast agent, a magnetic resonance imaging contrast agent and a fluorescence imaging agent.

The compartments of the bioreactor device may be housed within a device body configured suitable for implantation into a subject e.g. an immuno-isolating shell. Non-limiting examples of immuno-isolating shells include mechanical membranes, for example having straw or pouch configurations; synthetic membranes such as polyacrylonitrile/polyvinylchloride, polysulfone, cellulose acetate and hydroxyethylmethacrylate/methylmethacrylate.

An exemplary configuration of the present device is shown in FIG. I and is referred to herein as device 10.

Device 10 includes a vascular compartment (referred to hereinabove as the first compartment) 12 comprising a vascular prosthesis 14, a blood inport 16 and a blood outport 18. Blood inport 16 and blood outport 18 may be attached to a subject's vasculature. Device 10 also includes a cellular compartment (referred to hereinabove as the second compartment) 20 comprising a cell injection port 22 and a cell flushing port 24. Cell injection port 22 and a cell flushing port 24 allow cells and accompanying medium to be added and/or removed from device 10 following implantation. In addition, device 10 includes membrane 26 interposed between vascular compartment 12 and cellular compartment 20.

According to a preferred embodiment, vascular compartment 12 and cellular compartment 20 are situated within a housing 28.

As mentioned hereinabove, the bioreactor of the present invention can be utilized to treat conditions, especially chronic conditions, including, but not limited to, the treatment of diabetes, hemophilia, dwarfism, anemia, kidney failure, hepatic failure, immunodeficiency disorders, pituitary disorders, and central nervous system disorders. Nervous system disorders which may be treated include, but are not limited to, chronic pain, Parkinson's disease, Alzheimer's disease and amyotrophic lateral sclerosis.

Several configurations of the present device can be utilized for such treatment.

In a first configuration, the device is wholly implanted in any suitable location in the recipient. For example, the device may be implanted subcutaneously and/or peritoneally. Alternatively, the device may be implanted retroperitoneally, such as a bioartificial renal tubule. Typically insulin producing cells are implanted in the peritoneal cavity, although other cavities are also envisaged. In this way insulin may be secreted into the blood and delivered systemically and in addition insulin may be secreted into the portal system thereby delivering insulin directly to the liver. The advantage of such a dual secretion pathways ensures a better insulin/glucose control mechanism which in turn results in decreased side complications.

In a second configuration, the device is provided extracorporeally but still connected to a subject's vasculature. This is less invasive than whole implantation described above and may be particularly suitable for immunocompromised patients or for patients requiring a short-term treatment. It is also more readily accessible and therefore may be particularly useful for patients requiring ongoing addition and/or removal of cells.

Prior to, during and or following full or partial implantation of the device, the recipient may be treated with pharmaceutical agents to suppress his immune system. Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE^{R}), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

Following connection, the immunogenicity of the bioreactor may optionally be monitored. It has been found that there is a correlation between allograft failure and increased titers of antidonor HLA antibodies, measured by panel reactive antibody testing, thus this test may be used for monitoring immunogenicity of the bioreactor. Considerable progress in developing a molecular based diagnostic approach to define early markers for rejection has recently been made, and quantitative analysis of the genes involved in the cytolytic machinery of cytotoxic T-lymphocytes including granzyme B, perforin, and Fas ligand in the peripheral blood may be used as an approach to detect early episodes of rejection and drive anti-rejection therapy.

Cells are introduced into the bioreactor either following or prior to its connection to the recipients vasculature, preferably through the cell injection port. The cells are typically injected into the bioreactor together with an appropriate medium either prior to or following bioreactor implantation. The medium may comprise additional factors to aid in the maintenance and/or promote growth of the cells (e.g. nutrients, cell stabilizers, growth factors etc.).

Following cell implantation, the subject maybe examined periodically to assess the performance of the implant.

If the bioreactor comprising insulin secreting cells is implanted into a diabetic patient, the patient may be monitored periodically by measuring blood glucose and glycosylated hemoglobin HbAlc levels. The extent of mean fluctuations in serum glucose concentrations, measured as mean amplitude of glycemic variation in a 24-h period is a useful tool in the assessment of metabolic instability. In addition, or alternatively, basal and peak C peptide response to glucose stimulation may be examined. C-peptide and insulin are produced in equimolar amounts from the proinsulin molecule by pancreatic beta cells, and measurement of plasma C-peptide allows monitoring of beta cell function when the patient is treated with exogenous insulin.

Hemoglobin Alc HbAlc is formed from the irreversible nonenzymatic glycation of the hemoglobin beta chain, and is directly proportional to the ambient glucose concentration. The level of HbAlc directly correlates with blood sugar levels and lasts longer after the maximum blood sugar level is observed, making it a more reliable long-term marker of blood sugar level control than immediate glycemia measurement.

The extent of required exogenous insulin administration is also an accurate measure of the functioning of implanted insulin-producing cells. Metabolic tests such as oral and intravenous glucose tolerance tests can be performed that provide detailed information on the performance of the transplanted insulin-producing cells to secretagogue stimuli.

Depending on the results of the metabolic tests, new cells can be added either from the same population or an alternative population, old cells may be replaced or a combination of both. Old cells may be removed by flushing the bioreactor with a physiological medium e.g. saline. to As use herein the term “about” refers to ±10%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A bioreactor device comprising:
(i) a first compartment being configured capable of fluidic communication with a vasculature of a subject; and
(ii) a second compartment configured for containing cells, said second compartment being separated from said first compartment by a membrane.

2. The device of claim 1, wherein said membrane blocks passage of said cells from said second compartment to said first compartment.

3. The device of claim 1, wherein said membrane enables passage of fluids and molecules to and from said second compartment.

4. The device of claim 1, wherein said first and said second compartments are housed within a device body configured suitable for implantation into said subject.

5. The device of claim 1, wherein said first compartment comprises a blood inport and a blood outport.

6. The device of claim 5, wherein said first compartment further comprises a vascular prosthesis connected to each of said blood inport and said blood outport.

7. The device of claim 1, wherein said first compartment includes a cell injection port.

8. The device of claim 1, wherein at least one of said first compartment, said second compartment and said membrane is made of non-woven polymer fibers.

9. The device of claim 8, wherein said non-woven polymer fibers are electrospun polymer fibers.

10. The device of claim 1, wherein said first compartment and/or said membrane comprise at least one pharmaceutical agent.

11. The device of claim 10, wherein at least one pharmaceutical agent is impregnated in said vascular compartment and/or said membrane.

12. The device of claims 10, wherein said pharmaceutical agent is a therapeutic agent or a diagnostic agent.

13. The device of claim 12, wherein said therapeutic agent is selected from the group consisting of heparin, tridodecylmethylammonium-heparin, epothilone A, epothilone B, rotomycine, ticlopidine, dexamethasone and caumadin.

14. The device of claim 8, wherein said polymer fibers have a permeability cutoff at a molecular weight of about between 40 and 250 kilo Daltons.

15. A method of delivering a cell population into a subject in need thereof, the method comprising
(a) providing a device comprising:
(i) a first compartment being configured capable of fluidic communication with a vasculature of a subject; and
(ii) a second compartment configured for containing cells, said second compartment being separated from said first compartment by a membrane;
(b) connecting said device to said vasculature of said subject; and
(c) introducing the cell population into said second compartment, thereby delivering a cell population into a subject in need thereof.

16. The method of claim 15, wherein step (c) is effected prior to step (b).

17. The method of claim 15, wherein step (c) is effected following step (b).

18. The method of claim 15, wherein at least one of said first compartment and said second compartment is made of non-woven polymer fibers.

19. The method of claim 18, wherein said non-woven polymer fibers are electrospun polymer fibers.

20. The method of claim 15, wherein step (b) is effected by an end to end vascular connection.

21. The method of claim 15, wherein step (b) is effected by an end to side vascular connection.

22. The method of claim 15, wherein step (b) is effected by a combination of an end to end vascular connection and an end to side vascular connection.

23. The method of claim 15, wherein the cell population comprises an insulin secreting cell population.

24. The method of claim 15, wherein the cell population comprises Islets of Langerhans.
